(19) United States
(12) Patent Application Publication
(10) Pub. No.: US 2002/0193721 A1
(43) Pub. Date: Dec. 19, 2002

(54) Wound closure grid tape apparatus and method

(76) Inventor: Dean VanDruff, Dallas, TX (US)

Correspondence Address: Dean VanDruff, 14220 Pike Road, SARATOGA, CA, 95070, US

(21) Appl. No.: 09/882,640

(22) Filed: Jun. 15, 2001

(51) Int. Cl. A61F 13/00

(52) U.S. Cl. 602/41

(57) ABSTRACT

A wound closure tape apparatus and method shaped substantially like a grid or screen meant to replace or work in conjunction with traditional stitches and staples.

### BACKGROUND OF THE INVENTION

1. Technical Field

The present invention is directed to a medical wound closure grid tape. More specifically, the present invention is directed to an apparatus and method to seal wounds without traditional sutures or medical staples, or in conjunction with traditional sutures or medical staples.

2. Description of Related Art

Many benefits of existing Wound Closure Tape (WCT) are documented in the referenced patents. WCT allows for quick sealing of wounds and averts the trauma caused by puncturing the skin for the purpose of suturing or stapling. WCTs usually provide some novel means for porosity, and provide a balance between holding strength and a controlled ability to flex and drape upon application.

At present, WCT is mainly used in plastic surgery applications to reduce scarring and healing time. Existing WCT can also be used for minor wound care, but at present the preponderance of patients and medical practitioners still opt for traditional stitches. This is unfortunate, since compared to traditional stitching WCT has many advantages. Most obviously, WCT averts the further trauma of inflicting additional puncture wounds by stitching through the skin, avoiding the resultant bleeding, medium-term irritation, risk of infection, and scarring that result. As well, the surface nature of WCT has the effect of encouraging the wound into a more planar position than traditional sutures, reducing scarring along the wound line as well. Even with these advantages, WCT has yet to find its way into mainstream use, despite many innovations in materials and packaging.

Previous embodiments fall roughly into two categories. First are the porous material WCTs which use a permeable medium. Typically these are in strips which are applied one at a time across the wound. Examples are U.S. Pat. Nos. 3,908,650, 4,302,500, and 5,947,988. Second are the WCTs constructed via patterned openings in the tape, which patterns allows the tape to be flexible enough for drape and also to provide permeability. Examples are U.S. Pat. Nos. 3,991,754, 4,612,230, and 4,999,235.

An invention of note is U.S. Pat. No. 4,950,282, which holds multiple WCT strips together at one end in a unique comb-like configuration. This provides a means for quicker application of the WCT strips. Other than this patent, which has its own unique configuration, WCT is largely focused on the strips themselves, methods of porosity, materials, and the like.

Present WCT embodiments differ markedly from the present invention which is a novel overall configuration and defined shape. Some present WCTs may work well as materials within the present invention, but will be a subset of it.

A problem with both stitches and WCT is aligning both sides of the wound along its length during closure such that the wound will have a natural flow and drape when healed. U.S. Pat. No. 4,950,282 addresses this problem somewhat. Beyond this, what is needed is an ability to seal the wound all at once; or at least in larger sections than one stitch at a time, which tends to amplify small errors and stitch misalignments made early on.

The spacing and tightness of traditional stitches can have a large effect on residual scarring. Large spacing requires tighter sutures. Where sutures enter outside of the wound to cross it, at this point the seal is over-tight such that midway to the next suture the seal is not lost, and so on along the wound, where larger spaced sutures amplify this effect and leave more dramatic scarring. Such can be averted by using smaller sutures, closer spacing, careful alignment, and maintaining consistent tightness, but this takes considerably more time to achieve; and as a result, most people end up with unsightly scars. The situation is similar with most WCTs as with sutures, as each is normally applied individually. The present invention provides a substantial improvement in this regard, breaking through the time spent/scar-quality conundrum. By using the proper granularity of the present invention's “stitches” to be used, and given that it is all of one piece to begin with, getting the entire wound aligned and sealed with the proper tightness is made dramatically easier.

Existing WCT is meant principally for smaller wounds, and is not regarded as appropriate for larger wounds. But the need exists for rapid and secure closure for larger wounds as well, especially in the field instead of using tourniquets or external pressure to control bleeding. For example, a deep leg wound could be sealed with a large sheet of the present invention to contain bleeding and for protection from infection during transport to a suitable medical facility. On arrival, the tape could be removed for internal repairs and perhaps later another sheet re-applied to provide basic alignment of the skin as a template for traditional stitches, where the sutures would be woven through the grid, if needed; or the present invention may be the only means deemed required to close the wound.

Another problem with traditional stitches or staples on areas of the body where skin movement and stretch are high is that they do not extend very far away from the wound. As far as they do extend, a semi-rigid zone comprised of the tightened skin and the stitches serves to protect the wound area from normal skin stresses. Existing WCT strips can extend further away from the wound, but lack the structural tightness and protection of stitches in high-movement skin areas, so it is generally not used for fear that it will not hold the wound closed in such situations. With the present invention, the overall shape can be chosen to provide a semi-rigid safety zone around the wound that inhibits significant movement, and this area can extend well beyond the wound itself. This allows movement stresses to be borne by skin some distance away from the wound, obviating the need for over-tightening the wound. As well, the overall semi-rigid patch acts as an aversive stimulus to inappropriate movement. Any loss of adhesion, if it occurs, would start on the outside edges of the present invention first, where such failure is non-critical and serves as an early warning.

WCT is often a fairly thin tape, balancing strength with flexibility. The present invention uses its inherent structure to provide both in a much more controlled way over the entire area of the wound, and beyond. In the case of existing WCT, the flex-strength trade-off means that the strips need be quite a bit wider than the present invention, giving the effect of covering significantly more skin around the wound area than traditional stitching, a further impediment to its acceptance as a replacement to sutures.

In short, the flimsy, tape-like nature of existing WCT is a psychological barrier as compared to the obvious means of closure that traditional stitches provide. With the present invention, however, the tape can be made semi-rigid and to actually look a bit like stitches, and can seal even large wounds. Given this, it is felt that the present invention will not be perceived in the same way as existing WCT—as a delicate and highly specialized material and technique for use mainly by plastic surgeons—and begin to be used in general medical practice.

What is needed is a new kind of WCT tape that: 1) has a defined, easily recognized overall structure and functionality, 2) is perceived to be strong enough to be trusted as a valid replacement for traditional stitches, thus avoiding the additional skin puncturing of said stitches; 3) offers similar or better performance than existing WCTS; 4) can be used at home, in the field, or in a medical facility; 5) has a wide breadth of performance for various applications and sizes; and 6) is inexpensive. Given such, it is anticipated that such a WCT would become the first choice of medical practitioners for appropriate applications, and part of any standard First-Aid kit for quick wound closure in the field.

### SUMMARY OF THE INVENTION

The present invention provides an apparatus and method for a grid-like medical wound closure tape to replace traditional stitches or medical staples or aid in skin placement when used in conjunction with said stitches or staples.

### BRIEF DESCRIPTION OF THE DRAWINGS

The novel features believed characteristic of the invention are set forth in the appended claims. The invention itself, however, as well as an exemplary mode of use, further objectives and advantages thereof, will best be understood by reference to the following detailed description of an illustrative embodiment when read in conjunction with the accompanying drawings, wherein:

FIG. 1 is an exemplary drawing of a top view of a woven, screen-like exemplary embodiment of the present invention;

FIG. 2 is an exemplary drawing of a perspective side view of FIG. 1;

FIG. 3 is an exemplary drawing of a top view of a non-woven, screen-like exemplary embodiment of the present invention;

FIG. 4 is an exemplary drawing of a perspective side view of FIG. 3;

FIG. 5 is an exemplary drawing of a top view of a configuration much like FIG. 3, but using a rectangular cross-member rather than an oval or circular, as yet another exemplary embodiment of the present invention;

FIG. 6 is an exemplary drawing of a perspective side view of FIG. 5;

FIG. 7 is an exemplary drawing of a top view of a rectangular cross-membered monolithic grid as an exemplary embodiment of the present invention;

FIG. 8 is an exemplary illustration of a perspective side view of FIG. 7;

FIG. 9 is an exemplary illustration an arm wound;

FIG. 10 is an exemplary illustration showing how the present invention would look when applied to close the wound of FIG. 9, according to one exemplary embodiment of the present invention.

### DETAILED DESCRIPTION OF THE PREFERRED EMBODIMENT

The present invention is directed to a medical Wound Closure Grid Tape (WCGT). The major novelty of the present invention resides in its overall grid or screen-like configuration. The terms “grid” and “screen” are meant to be descriptive of the tape's appearance when viewed from above, not to limit whether it is woven, nonwoven, or monolithic. With this description in view, along with further detail provided in the claims, the structure of the present invention will herein be referred to simply by the term “grid”.

“Wound” herein refers to any type of skin laceration, including surgical incisions.

WCGT is meant to replace traditional stitches, or aid as a template for aligning skin for traditional stitches.

In grid spacing, WCGT structure can be roughly described as ranging in aperture from that of a door screen (around 2 millimeters) to that of rabbit-cage (around 10 millimeters) or wider, and not necessarily square, where different applications would require different spacing granularities. In any case, the present invention is more akin to a screen or grid than fabric or cloth. The open area of WCGT will generally be greater than 50%, and considerably more so in most embodiments. An exemplary, typical sizing for general purpose WCGT is a 5 millimeter grid made up of ½ millimeter cross-members, resulting in about a 90% open area. Thus, WCGT is breathable and porous by overall structure, and may or may not be breathable in the material used in its cross-members. With small enough cross-members, porosity is not critical as the WCGT performs much like traditional sutures.

The present invention is meant to provide external stitches in about the same (or slightly smaller) spacing as traditional stitches that are held in place by cross-members and adhesion to the skin. The “stitch” then, can be thought of as being planar to the skin and deriving its strength to hold the wound closed from adhesion by cross-members at right angles to the wound, and also from the circuits created by the cross-members of the grid structure rather than by looping into and through the skin surface.

In stiffer embodiments of WCGT, the support from the surrounding structure can be thought of as a stitch-laid-flat along the surface of the skin, connected to other “stitches” in a matrix which shares components and gripping strength. Each “stitch” element that overlaps the wound is held secure by surrounding grid elements and also the adhesion moving away from the wound at right angles on either side. By such means, a patch area around the wound can be custom sized as would be appropriate, creating a boundary zone around the wound to protect it from separation due to skin movement during healing.

The structure itself is made up of cross-members of any suitable material, and the adhesive is any suitable medical adhesive. In other words, the present invention is not material or adhesive dependent. Materials and adhesives used in WCGT may be covered by existing or future patents, or be in the public domain. The referenced patents discuss the properties required of such materials in non-grid applications. Using the unique structure of the present invention, materials may need to be stiffer overall and the adhesive stronger, since there will be less material used.

The adhesive may be of a kind that can achieve and maintain adhesion in the presence of blood and/or water. The adhesive may also be of a kind that can be neutralized by a catalyst such as alcohol for later removal. Typically, removal of the bandage would be along the wound rather than across it, which illustrates another structural advantage of the present invention over existing WCT.

As with traditional stitches, WCGT can be covered with bandages, gauze, etc., which may or may not be part of the WCGT package. Cleansing of the wound can be done without removal of the WCGT, similar to traditional stitches, as long as the fluids used do not neutralize the adhesive; or, the WCGT can be removed and replaced for this purpose. As well, antibiotics can be applied through the WCGT structure as long as they are selected so as not to weaken the adhesive.

For larger wounds; wounds that require precision alignment; or wounds on parts of the body where it is felt traditional stitches are needed, WCGT can be used to initially seal and align the wound as a template for traditional stitches or medical staples. In this case, the adhesive may be much less strong so that the wound can be easily repositioned by multiple detachments and reattachments. After the traditional stitching or stapling is done through the WCGT grid, it may then be removed after being clipped across any intersecting points needed. Or, in the case where a stronger adhesive is used, the tape may remain to provide a protected healing zone around the stitches, or added later for this purpose.

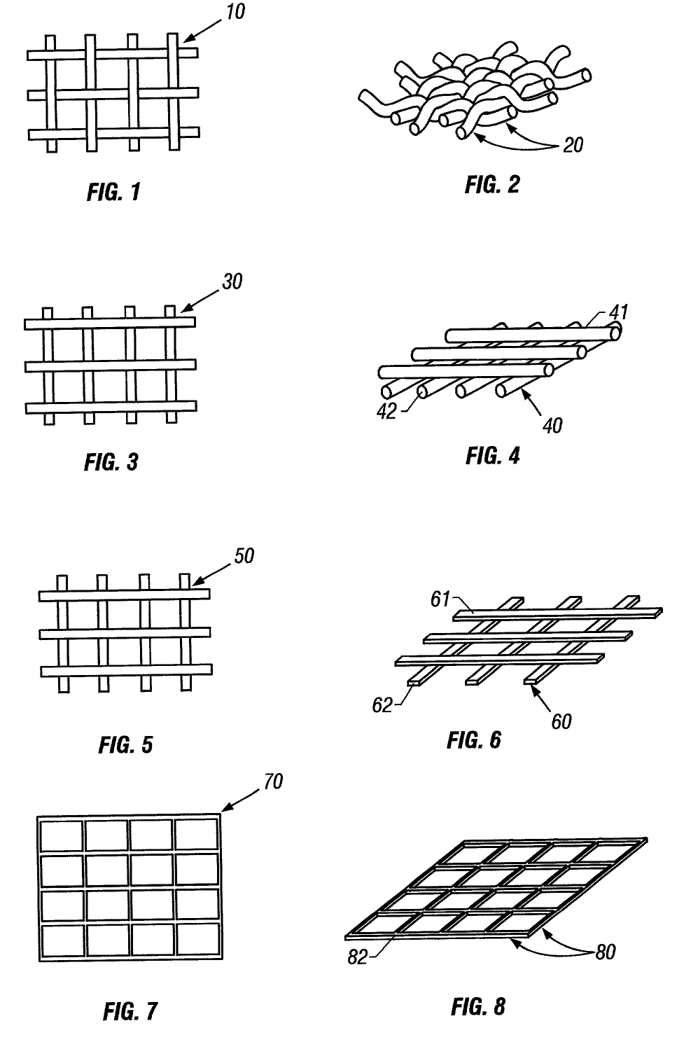

FIG. 1 is one exemplary drawing of the present invention, which is further depicted in perspective side view in FIG. 2. This pair of illustrations shows a screen-like, woven exemplary embodiment of the present invention. Intersection points 10 may or may not be bonded, preferably not to gain the advantage of flexible drape over complex curved surfaces. In this embodiment, adhesive surface contact points 20 will not be in contiguous, linear contact with the skin. This may be a limitation in certain applications, but also an advantage in environments of rough skin surfaces; and/or where hair around the wound cannot be removed; and/or where it is otherwise desired that contiguous contact lines be avoided. As well, for highly curved wounds or for multiple lacerations that need to be sealed within an area, the dispersed adhesive contact points of this embodiment are less direction dependent. The most obvious disadvantage of this configuration is the lack of a consistent adhesion surface and the difficulty of applying the adhesive 20 to the bottom of the grid.

FIG. 3 is another exemplary drawing of the present invention, which is further depicted in perspective side view in FIG. 4. This pair of illustrations shows a non-woven, screen or grid-like exemplary embodiment of the present invention where the cross-member elements going in each direction are largely coplanar, and bonded at each intersection 30. The cross-member elements in this embodiment mimic traditional stitches in appearance, being round 42. The non-woven configuration has the advantage of allowing a contiguous adhesion surface on the bottom parallel elements 40. The bottom adhesive elements can then be placed across the wound, similar to traditional WCT, where the right angle cross-members on top 41 provide alignment and further structural support for the wound area. Alternately, if there is reason to keep adhesive off of the wound, the glued bottom members 40 could be placed in the direction of, or parallel with a substantially straight wound where the top members 41 would bridge the wound and hold it closed by the positioning of the members surrounding 40 and just alongside. This parallel configuration might be useful to temporarily seal a wound where it is anticipated that the wound will be opened again in the near future, so as to avoid adhesion of the adhesive elements 40 to the traumatized tissue; or in the case where the WCGT is being used to hold a wound closed and in proper position as a template for traditional suturing. For this purpose the top elements 41 may be spaced further apart than those underneath, perhaps several times as far. The material used may be chosen to flex and stretch enough to provide appropriate drape, while still being strong enough to hold the wound closed. Other example variations are to make the top elements 41 smaller in size than the bottom elements 40, or to make the top elements 41 a different geometric shape than the bottom 40, or to make the top elements 41 a different stiffness or material than the bottom elements 40.

FIG. 5 is an exemplary drawing of the present invention, which is further depicted in perspective side view in FIG. 6. This pair of illustrations shows a non-woven, screen or grid-like exemplary embodiment of the present invention where the cross-member elements going in each direction 60, 61 are largely coplanar, and where the cross-member elements are rectangular 62 rather than round or oval 42 as in FIGS. 1 through 4; otherwise it is very similar in configuration to FIGS. 3 and 4. As in FIGS. 3 and 4, the intersection of the cross-members 60 are bonded. FIGS. 5 and 6 are included to illustrate that any shaped cross-member element 62 is within the scope of this invention, as well as to discuss the merits of square or rectangular cross-members beyond the previous discussion of the non-woven configuration provided for FIGS. 3 and 4. Specifically, a square or rectangular cross-member 62 has the advantage of providing more adhesion surface to the skin 60, but this might also be a disadvantage due to slightly higher skin coverage and the potential for more difficult removal. To compensate for these two disadvantages, it is possible that the elements 60, 61 in this configuration may be constructed of porous material. Or, alternately, if the elements are substantially solid, to use a less tacky adhesive 60 and a square rather than the rectangular shape 62 as shown.

FIG. 7 is an exemplary drawing of the present invention, which is further depicted in perspective side view in FIG. 8. This pair of illustrations shows a rectangular cross-membered monolithic 70 grid as an exemplary embodiment of the present invention. The advantages and disadvantages of the squarish or rectangular cross-membered elements 82 were discussed with FIGS. 5 and 6. Of note here is that a monolithic grid can provide adhesion on the bottom surface of all members 80, and has unidirectional features mentioned in FIGS. 1 and 2 that make it suitable for curved or multiple wound closure situations.

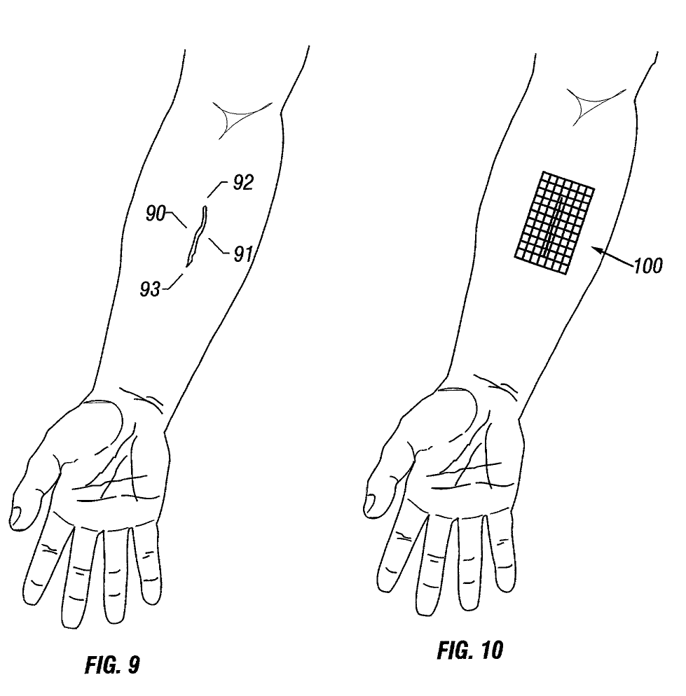

FIG. 9 is an exemplary illustration of an arm wound;

FIG. 10 is an exemplary illustration showing how the present invention would be used to secure the wound of FIG. 9, according to one exemplary embodiment 100 of the present invention. By way of further explanation, and assuming a flexible but semi-rigid embodiment, three exemplary techniques of wound closure will be described. The first exemplary means of sealing the wound is to place the WCGT 100 on one side of the wound 90 with a slight bend along the axis between numbers 92 and 93 such that the other side 91 is tilted above the skin and thus will not adhere. With side 90 pressed in place and with the appropriate pressure applied, the WCGT is tugged with one hand while the other hand (or the thumb of the hand if being self-applied or with one hand) pushes the skin on the other side 91 into position, and then the WCGT is lowered into place. This handling can be done by grasping the WCGT at the edges where denigration of the adhesive is not as critical, or alternately by leaving the backing attached to the secondary side 91 while the first side is pressed into place 90. The second exemplary means to apply is to curve the WCGT upwards, or hold it in a curved position upwards along the longer axis shown, and position it starting at one end of the wound 92. Then, as previously, the wound is pressed together and the WCGT is pressed into place gradually along the length of the wound, going from one end 92 to the other 93. For small wounds, a third approach is to hold the WCGT by the edges and close the wound via pressures outside of the expected WCGT area, and then press the WCGT in place all at once.

The present invention provides a significant improvement in regard to overall alignment of the lacerated surfaces, as the entire wound—or sections of it—can be situated and the WCGT placed in one quick motion rather than one stitch at a time from side to side; or crudely at points along the way and then filled in later. The whole wound can be seen and fixed at once, or sections of it for larger wounds, or quickly along the wound as the tape is rolled on. If a misalignment occurs the WCGT can be removed and replaced with significantly less trauma than with other techniques.

In field situations, WCGT can be used to quickly seal wounds to control bleeding and to allow for transport. One of the major advantages to the WCGT structure is its scalability and utility from small wounds to large. A large, nearly wire-like stiffness WCGT with a strong adhesive can be bent into rough shape while the backing is still on, then applied within a few seconds to patient in the field. Alternatively, for small home cuts, a flexible fabric-like WCGT can seal minor wounds before going to a medical facility; or perhaps as the primary and only means needed for closure.

The present invention is not package dependant, but certain packaging embodiments are preferable. Obviously, the WCGT and packaging need to be sterile. Example embodiments would be packaging for various sizes in flat sheets that can be cut or torn to fit; flat packages with backing that can be removed in sections for handling purposes, and rolls of backed tape either in separate rolls or in a dispensing mechanism, in various widths. In the case of separate rolls, a suggested embodiment is to have the adhesive side facing outwards so as to allow a “rolling on” along wounds based on the natural curvature of the roll.

The present invention is not color dependant, and may be colored to convey certain size and application distinctions. It may also be colored for marketing purposes with designs, logos, etc.

The present invention makes for a viable alternative to traditional means of closing wounds, allowing patients and medical practitioners to avoid the further trauma of stitching through the skin with traditional stitches, or puncturing the skin with medical staples.

The present invention is inexpensive to manufacture. It also allows for very fast sealing of wounds, a significant benefit for field applications as well as in medical facilities.

In situations of minor wounds where the injury is only topical, WCGT may be applied without the time and expense of a medical facility visit; or if such a visit were necessary, applied on location before the trip to the medical facility rather than applying a tourniquet, pressure with a soak towel alone, or the like.

While the present invention is described and shown in the preferred embodiments depicted, it should be apparent by those skilled in the art that other embodiments not shown would be within the spirit and scope of this invention. The description of the present invention has been presented for purposes of illustration and description, but is not limited to be exhaustive or limited to the invention in the forms disclosed. The embodiment was chosen and described in order to best explain the principles of the invention, the practical application, and to enable others of ordinary skill in the art to understand the invention for various embodiments with various modifications as are suited to the particular use contemplated. While the invention has been particularly shown and described with reference to exemplary preferred embodiments, it will be understood by those skilled in the art that various changes in form and detail may be made therein without departing from the spirit and scope of the invention.

What is claimed is:

1. An apparatus of wound closure grid tape comprising:
a shape, if viewed from above, in substantially a grid pattern;
wherein the tape elements are spaced between 1 millimeter to 20 millimeters in one direction, and 1 millimeters to 50 millimeters in the other, matching or not;
wherein the resultant open areas are substantially square or rectangular; and
wherein the cross-members of said tape act as external stitches for the wound.

2. The apparatus of claim 1, wherein the grid tape structure is constructed of any suitable material.

3. The apparatus of claim 1, wherein the grid tape is comprised of elements that in cross-section can be made in any geometric shape; such as: round or oval or square or rectangular; and wherein grid elements going in one direction may differ from the other.

4. The apparatus of claim 1, wherein the grid tape is at least partially coated on one side with any non-irritating pressure sensitive medical adhesive.

5. The apparatus of claim 1, wherein the materials used to construct the grid tape have controlled elastic qualities, being able to give a certain amount and then no more until torn.

6. The apparatus of claim 1, wherein the grid tape bottom surface is protected before use by a removable backing strip.

7. The apparatus of claim 1, wherein the grid tape is sterilized and/or is in sterile packaging.

8. The apparatus of claim 1, wherein grid tape segments are placed on removable backing sheets.

9. The apparatus of claim 1, wherein the grid tape is packaged in a roll format.

10. The apparatus of claim 1, wherein the grid tape is packaged in a roll format, where the adhesive side is facing outwards.

11. The apparatus of claim 1, wherein the grid tape has been packaged in a dispensing mechanism.

12. The apparatus of claim 1, wherein the grid tape grid elements are sufficiently strong to hold the wound closed, but weak enough—or scribed or otherwise made to be weak enough—to be torn.

13. The apparatus of claim 1, wherein the grid tape is one of the layers of an apparatus comprised of other layers as well, such as a gauze pad or other protective or structural member.

14. The apparatus of claim 1, wherein the tape does not have adhesive on one side, but can be taped-through with another tape placed on top, or alternatively can be adhered by applying adhesive directly to the skin around the wound.

15. A method of wound closure grid tape comprising:
a shape, if viewed from above, in substantially a grid pattern;
wherein the tape elements are spaced between 1 millimeter to 20 millimeters in one direction, and 1 millimeters to 50 millimeters in the other, matching or not;
wherein the resultant open areas are substantially square or rectangular; and
wherein the cross-members of said tape act as external stitches for the wound.

16. The method of claim 15, wherein the grid tape structure is constructed of any suitable material.

17. The method of claim 15, wherein the grid tape is comprised of elements that in cross-section can be made in any geometric shape; such as: round or oval or square or rectangular; and wherein grid elements going in one direction may differ from the other.

18. The method of claim 15, wherein the grid tape is at least partially coated on one side with any non-irritating pressure sensitive medical adhesive.

19. The method of claim 15, wherein the materials used to construct the grid tape have controlled elastic qualities, being able to give a certain amount and then no more until torn.

20. The method of claim 15, wherein the grid tape bottom surface is protected before use by a removable backing strip.

21. The method of claim 15, wherein the grid tape is sterilized and/or is in sterile packaging.

22. The method of claim 15, wherein grid tape segments are placed on removable backing sheets.

23. The method of claim 15, wherein the grid tape is packaged in a roll format.

24. The method of claim 15, wherein the grid tape is packaged in a roll format, where the adhesive side is facing outwards.

25. The method of claim 15, wherein the grid tape has been packaged in a dispensing mechanism.

26. The method of claim 15, wherein the grid tape grid elements are sufficiently strong to hold the wound closed, but weak enough—or scribed or otherwise made to be weak enough—to be torn.

27. The method of claim 15, wherein the grid tape is used in conjunction with traditional stitches or staples, where the grid tape is used to align the wound and hold it in place beforehand.

28. The method of claim 15, wherein the grid tape is used in conjunction with traditional stitches or staples, where the grid tape is left on after being used as a template or applied afterwards for additional support and protection.

29. The method of claim 15, wherein the grid tape is one of the layers of an apparatus comprised of other layers as well, such as a gauze pad or other protective or structural member.

30. The method of claim 15, wherein the tape does not have adhesive on one side, but can be taped-through with another tape placed on top, or alternatively can be adhered by applying adhesive directly to the skin around the wound.
